(19) United States
(12) Patent Application Publication
(10) Pub. No.: US 2013/0171078 A1
(43) Pub. Date: Jul. 4, 2013

(54) Transfer Resistant Cosmetic Composition

(71) Applicant: Clement Lawson, Bay Shore, NY (US)

(72) Inventor: Clement Lawson, Bay Shore, NY (US)

(21) Appl. No.: 13/537,304

(22) Filed: Jun. 29, 2012

Related U.S. Application Data
(60) Provisional application No. 61/502,915, filed on Jun. 30, 2011.

(51) Int. Cl. A61K 8/895; A61Q 17/04; A61Q 1/06

(52) U.S. Cl. CPC A61K 8/895; A61Q 1/06; A61Q 17/04
USPC 424/59; 424/78.26

(57) ABSTRACT

The present invention relates to a transfer-resistant cosmetic composition comprising at least one silicone film forming polymer derived from acrylic acid, at least one naturally derived volatile solvent and a cosmetically acceptable carrier.

### CROSS-REFERENCE TO RELATED APPLICATIONS

The present application claims priority from U.S. Provisional Application No. 61/502,915, filed Jun. 30, 2011.

### FIELD OF THE INVENTION

The present invention relates generally to color cosmetics having a reduced propensity to transfer or rub-off during wear, and which may be thickened before addition of secondary thickening agents. More particularly, the invention relates to color cosmetics comprising copolymers of silicone and ethylenically unsaturated monomers such as acrylic acid, methacrylic acid or their simple esters in combination with an organic solvent system.

### BACKGROUND OF THE INVENTION

The ability of a cosmetic product to remain on a surface (e.g., skin, lips, hair, eyelashes, etc.) when that surface comes into contact with another surface is commonly referred to as “transfer resistance.” Ideally, a cosmetic film should last until the consumer wants to remove it by washing with water or using remover compositions. However, many cosmetics are deficient in this regard and readily transfer to the fingers, napkins, clothing, utensils, cups, and the like. This problem is particularly disadvantageous with color cosmetics, such as lipsticks, foundations, and mascara, where clothing can become discolored on contact and the cosmetic must be frequently re-applied to maintain a fresh appearance. Thus, much effort has been directed to developing so-called transfer-resistant cosmetics.

Transfer-resistant cosmetics typically employ a film forming polymer to provide the transfer resistant film on the skin, lips, hair or lashes. The class of polymers known as organosiloxanes, including polydimethylsiloxane (PDMS or Dimethicone), are well known for use in cosmetics due to their many desirable properties such as film formers, excellent spreading properties and biological inertness. More recently, the properties of silicone polymers have been modified by copolymerization with other organic monomers or polymers, such as polyurethanes, ethylenically unsaturated monomers or polymers thereof, and the like.

For example, in U.S. Patent Pub. 2008/0019932, color cosmetic compositions comprising at least one silicone film forming polymer, at least one pigment, and at least one dispersant that aids in dispersion of the pigment and silicone film forming polymer in the composition are described. The silicone film forming polymer may be, among others, a silicone acrylate. Silicone acrylate is typically dissolved in one of various volatile solvents such as isododecane. But in order to develop suitable compositions, such as lipsticks, additional thickeners such as waxes must be added to the matrix.

There is a need for color cosmetics which exhibit a diminished propensity to transfer or rub-off once applied to the skin, lips, or hair or a user and which exhibit longer wear than the presently available products. Additionally, there is a need for such a cosmetic which may be thickened without the necessity of secondary thickening agents.

### SUMMARY OF THE INVENTION

The present invention relates to a transfer-resistant cosmetic composition comprising at least one copolymer of silicone and ethylenically unsaturated monomers, more specifically ethylenically unsaturated monomers which may be acrylic acid, methacrylic acid or their simple esters and at least one vegetable oil derived volatile solvent and a cosmetically acceptable carrier.

### DETAILED DESCRIPTION OF THE INVENTION

The compositions of the invention comprise a copolymer of silicone and one or more ethylenically unsaturated monomers, such as acrylic acid, methacrylic acid or their simple esters and at least one volatile solvent derived from vegetable oil.

By derived from acrylic acid is meant that the polymers are the reaction products of monomers which include unsaturated carboxylic acid or carboxylate groups, for example, acrylic acid monomers, esters of acrylic acid monomers (acrylates), alkyl-substituted acrylic acid and/or acrylates, and the like, as well as block or graft copolymers comprising such film forming polymer derived from acrylic acid. The term (alkyl)acrylate is meant to include polymers and copolymers of acrylic acid monomers or esters of acrylic acid monomers.

All percentages, parts and ratios are based upon the total weight of the compositions of the present invention, unless otherwise specified. All such weights as they pertain to listed ingredients are based on the active level and therefore do not include solvents or by-products that may be included in commercially available materials, unless otherwise specified.

All molecular weights as used herein are weight average molecular weights expressed as grams/mole, unless otherwise specified.

As used herein, the term “volatile” refers to solvents having a boiling point at one atmosphere of 260° C. or less, preferably 250° C. or less, more preferably 230° C. or less, most preferably 225° C. or less. In addition, the boiling point for the volatile solvent will generally be at least about 50° C. preferably at least about 100° C. The term “nonvolatile” shall refer to solvents which have a boiling point at one atmosphere of greater than 260° C. The solvent should also be acceptable for topical application to the hair and skin (i.e., no undue irritation, sensitization or other reactions are induced by the solvent).

The term “polymer” as used herein shall include materials whether made by polymerization of one type of monomer or made by two (i.e., copolymers) or more types of monomers.

The term “water soluble” as used herein, means that the polymer is soluble in water. In general, the polymer should be soluble at 25° C. at a concentration of 0.1% by weight of the water solvent, preferably at 1%, more preferably at 5%, more preferably at 15%.

### Film Forming Polymer

The compositions comprise one or more copolymers of silicone and ethylenically unsaturated monomers such as acrylic acid, methacrylic acid or their simple esters. The film forming polymers may be natural or synthetic, or a combination of both, and may be in the form of solids, semi-solids, or liquids. The film forming polymer may be neutral or ionic in character, e.g. anionic, cationic, nonionic, or amphoteric.

Synthetic Polymers

Suitable synthetic polymers include homopolymers, copolymers, and block and graft copolymers comprised of repeating monomers such as acrylic or methacrylic acid or esters thereof, urethanes, esters, amides, styrene, vinyl, silicon, and so on. The synthetic polymers may be present in the composition in ranges from 0.1-95%, preferably 1-85%, more preferably 3-45% by weight of the total composition.

Examples of synthetic film forming polymers include those set forth in the CTFA Cosmetic Ingredient Dictionary and Handbook, Eighth Edition, 2000, pages 1744 through 1747.

a. Silicone Resins

Cross-linked silicones, also known as silicone resins, are suitable for use in the compositions and method of the invention. Preferred silicone resins have the general formula:

[(RR′R″)₃SiO_{1/2}]ₓ[SiO₂]_{y},

wherein R, R′ and R″ are each independently a C₁₋₁₀ straight or branched chain alkyl or phenyl, and x and y are such that the ratio of (RR′R″)₃SiO_{1/2}units to SiO₂units is 0.5 to 1 to 1.5 to 1.

Preferably R, R′ and R″ are a C1-6alkyl, and more preferably are methyl and x and y are such that the ratio of (CH₃)₃SiO % units to SiO₂units is 0.75 to 1. Most preferred is this trimethylsiloxy silicate containing 2.4 to 2.9 weight percent hydroxyl groups which is formed by the reaction of the sodium salt of silicic acid, chlorotrimethylsilane, and isopropyl alcohol. The manufacture of trimethylsiloxy silicate is set forth in U.S. Pat. Nos. 2,676,182; 3,541,205; and 3,836,437. Trimethylsiloxy silicate as described is available from Momentive Performance Materials under trade name SR1000, or, if desired in a blend of trimetnhylsiloxysilicate and volatile silicone from Dow Corning Corporation under the trade name 749 Fluid which contains about 40-60% volatile silicone and about 40-60% trimethylsiloxy silicate.

b. Copolymers of Silicone and Organic Monomers

Also suitable a film forming polymers are copolymers of silicone and various organic, ethylenically unsaturated monomers, and optionally other monomers. Examples of such polymers are disclosed in U.S. Pat. No. 6,033,650. Preferred examples of these polymers include graft or block copolymers comprised of silicon moieties and C₁₋₁₂ alkyl acrylate or methacrylate monomers which may be substituted with one or more groups such as halogen or hydroxy, also referred to as silicone/acrylate copolymers. Suitable silicone acrylate copolymers may be purchased from 3M Company under the trade names VS-70 and SA-70, or from Shin Etsu Silicones.

Particular examples of suitable silicone acrylate copolymers include, without limitation, those having the INCI names Butyl Acrylate/Hydroxypropyl Dimethicone Acrylate Copolymer (CTFA Monograph ID 12998), Acrylates/Dimethicone Copolymer (CTFA Monograph ID 10082), Acrylates/Ethylhexyl Acrylate/Dimethicone Methacrylate Copolymer (CTFA Monograph ID 16592), and combinations thereof, etc. In a preferred embodiment, the acrylate film former selected from the group consisting of Butyl Acrylate/Hydroxypropyl Dimethicone Acrylate Copolymer (CTFA Monograph ID 12998), Acrylates/Dimethicone Copolymer (CTFA Monograph ID 10082), and combinations thereof.

c. Urethane Homo- and Copolymers

Also suitable are homo and copolymers of urethane. Homopolymers of urethane are often sold in an aqueous dispersion from vendors such as Alloid Colloids, B.F. Goodrich, and the like. Suitable urethane copolymers may be comprised of urethane monomers copolymerized with organic compounds, or other synthetic monomers.

d. Amides and Amines

Also suitable are various synthetic polymers containing amide or amine substituent groups. Examples of such polymers include nylon, ammonium polyacrylate, acrylamides copolymer, acrylates/acrylamide copolymers, acrylates ammonium acrylate copolymer, acrylates C₁₀₋₂₀ alkyl acrylate cross polymer, acrylates/carbamate crosspolymer, acrylates ceteth-20 itaconate copolymer, acrylates/dimethylaminoethyl methacrylate copolymer, ammonium acrylates copolymer, ammonium polyacrylate, ammonium styrene/acrylates copolymer, ammonium vinyl acetate/acrylates copolymer, aminomethylpropanol/acrylates/dime-thylaminoethylmethacrylate copolymer, and so on.

e. Other Synthetic Polymers

Other suitable synthetic polymers are comprised of one or more monomers selected from the following general formula:

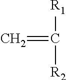

wherein R₁ is H, a C₁₋₃₀ straight or branched chain alkyl, aryl, aralkyl; R₂ is a pyrrolidone, or a substituted or unsubstituted aromatic, alicyclic, or bicyclic ring where the substitutents are C₁₋₃₀ straight or branched chain alkyl, or COOM wherein M is H, a C₁₋₃₀ straight or branched chain alkyl, pyrrolidone, or a substituted or unsubstituted aromatic, alicylic, or bicyclic ring where the substitutents are C₁₋₃₀ straight or branched chain alkyl which may be substituted with one or more halogens.

The synthetic polymers may comprise polar monomers such as acrylic acid or methacrylic acid, in combination with C₁₋₆ esters thereof. Most preferred is a synthetic polymer which comprises monomers of butyl methacrylate and acrylic acid.

Natural Polymers

A variety of natural polymers, or derivatives thereof are suitable, including cellulosics, chitins, chitosans, shellac, rosins, resins, animal or vegetable proteins and polypeptides, and so on. The natural polymers may be present in ranges from 0.1-95%, preferably 1-85%, more preferably 3-45% by weight of the total composition.

a. Cellulosics

Examples of suitable cellulosic polymers include nitrocellulose, mono- or diesters of cellulose formed by the reaction of cellulose with various organic acids, for example straight or branched chain carboxylic acids having from one to twenty, preferably one to ten carbon atoms, which may be substituted with one or more hydroxyl groups, Examples of such cellulosics include cellulose acetate, cellulose acetate isobutyrate, cellulose acetate propionate, cellulose acetate propionate carboxylate. Also suitable are cellulose polymers prepared by reacting with groups such as hydroxyl, alkoxyalkyl, hydro-xylalkyl where the alkoxyalkyl and alkyl groups have from about one to ten carbon atoms. Examples of such polymers are carboxylmethyl hydroxyethylcellulose, carboxymethylcellulose, ethyl cellulose, hydroxyethylcellulose, methyl ethylcellulose, hydroxypropylcellulose, hydroxylbutyl cellulose, hydroxybutyl methylcellulose, and so on.

b. Chitin or Chitosan

Chitins, or chitosan and derivatives thereof are also suitable natural film forming polymers for use in the compositions and method of the invention. Chitin is defined as a polysaccharide derived from the exoskeleton of marine invertebrates which contains chiefly N-acetyl-glucosamine residues. Chitosan is chitin that has been deacetylated. Both polymers may be used as is, or esterified to form mono-, di-, or triesters by reacting with various straight or branched chain organic acids having from one to thirty carbon atoms, alpha or beta hydroxy acids, or di- or tricarboxylic acids. Examples of chitin or chitosan esters include chitosan adipate, chitosan ascorbate, chitosan formate, chitosan glycolate, chitosan lactate, chitsan PCA, chitosan salicylate, chitosan succinamate, and so forth. Also suitable are simple derivatives of chitin or chitosan, which are formed by substituting moieties such as hydroxyl, C1-6alkoxy, and the like on the polymer. Examples of such derivatives include carboxylbutyl chitosan, carboxylmethyl chitosan, carboxyethyl chitosan, carboxylbutyl chitosan, and so on.

c. Proteins

Also suitable as film forming polymers are various animal and vegetable proteins including hydrolyzed animal protein, albumin, serum albumin, hydrolyzed wheat protein, hydrolyzed soy protein, hydrolyzed animal collagen, and mixtures thereof.

d. Dextrans

Also suitable are dextrans and alkoxy, or alkoxylalkyl derivatives thereof such as carboxymethyl dextran, carboxylethyl dextran, and so on.

e. Rosins, Resins and Gums

Also suitable are various natural resins and rosins and derivatives thereof such as Balsam Canada resin, hydrogenated rosin, glycol rosinate, shellac, and the like. Various gums are also suitable including acacia gum, and similar materials.

It may be desirable to have more than one film forming polymer in the composition. They polymers may be a combination of one or more synthetic polymers, or one or more natural polymers, or mixtures of both.

Generally, the film former is present in an amount from about 0.1% to about 85% by total weight of the composition. Typically, the film former is present from about 1% to about 75% by weight, more typically between about 5% and about 50%, and preferably, between about 10% and about 45% by weight, based on the total weight of the composition. These ranges also apply to combination of two or more different film formers.

### Solvent System

Film forming polymers discussed herein are typically solvated in a volatile solvent, such as methyl trimethicone, isododecane, dimethylsiloxane, cyclodimethicone pentamer/hexamer, or the like. These solutions are generally thin and flowable, so additional thickeners are added to formulate systems which are easily applied to the skin or hair.

It has been surprisingly found that film forming polymers, and particularly silicone acrylate polymers, form thickened systems when dissolved in volatile hydrocarbon solvents derived from vegetable oil or other sustainable feedstocks (naturally derived). Preferred solvents are volatile alkanes or esters sourced from vegetable oils such as coconut oil or palm oil. Specifically, coconut alkane (and) coco-caprylate/caprate (INCI name) is a preferred solvent for forming thickened cosmetic systems including silicone acrylate polymers. Such systems are contemplated to impart both water and oil transfer resistance. Furthermore, they are capable of incorporating shine enhancers to form shiny transfer-resistant compositions. The coconut alkane (and) coco-caprylate/caprate solvent is commercially available from, for example, Grant Industries, under the trade name Vegelight 1214LC. Other naturally derived solvents such as soy methyl ester, sold under the trade name SoyGuard® may also be included.

The compositions obtained in accordance with the invention do not have a brittle texture. Rather, they can be removed easily with the finger by adherence to it and they do not have a stringy texture; they do not flow in a viscous stream as does, for example, liquid honey.

In one embodiment, the silicone acrylate polymers are formed in a pre-mix with the naturally derived volatile solvent. As used herein, the term “pre-mix” means that the ingredients are combined together prior to their combination with other ingredients contained in the cosmetic composition.

The naturally derived volatile solvent is present in the amount of from about 5% to about 50%, more preferably from about 15% to about 40%, and most preferably from about 25% to about 30% by weight of the cosmetic composition.

Given the unique rheological benefits achieved by combining silicone acrylate polymers with the volatile solvent, other solvents traditionally used as silicone acrylate solvents are generally obviated or may degrade performance of the composition. Therefore, solvents selected from the group consisting of methyl trimethicone, isododecane, dimethylsiloxane, and cyclodimethicone pentamer/hexamer may be present, cumulatively, at levels of less than about 10%, preferably less than about 5%, and most preferably the compositions are substantially free of these solvents. As used herein, the term “substantially free” means that the ingredients are either absent from the composition or they are only present in trace amounts.

### Particulate Colorant Component

The composition may include a particulate colorant. As used herein, the term “colorant” generally refers to a color extender, dye, pigment, lake, toner, other agent, or a combination thereof, used to impart a color to a material, and includes inorganic, organic, water-soluble and water-insoluble substances.

The colorant may comprise, for example, an inorganic pigment. Exemplary inorganic pigments include, but are not limited to, metal oxides and metal hydroxides such as magnesium oxide, magnesium hydroxide, calcium oxide, calcium hydroxides, aluminum oxide, aluminum hydroxide, iron oxides, red iron oxide, yellow iron oxide, black iron oxide, iron hydroxides, titanium dioxide, titanium lower oxides, zirconium oxides, chromium oxides, chromium hydroxides, manganese oxides, cobalt oxides, cerium oxides, nickel oxides and zinc oxides and composite oxides and composite hydroxides such as iron titanate, cobalt titanate and cobalt aluminate. Non-metal oxides such as alumina and silica, ultramarine blue (i.e., sodium aluminum silicate containing sulfur), Prussian blue, manganese violet, bismuth oxychloride, talc, mica, sericite, magnesium carbonate, calcium carbonate, magnesium silicate, aluminum magnesium silicate, silica, titanated mica, iron oxide titanated mica, bismuth oxychloride, and the like, are also contemplated to be suitable inorganic pigments.

The colorant may comprise, for example, an organic pigment. Organic pigments can include, but are not limited to, at least one of carbon black, carmine, phthalocyanine blue and green pigment, diarylide yellow and orange pigments, and azo-type red and yellow pigments such as toluidine red, litho red, naphthol red and brown pigments, and combinations thereof.

The colorant component may comprise, for example, one or more dyes, toners or lakes. Lakes generally refer to a colorant prepared from a water-soluble organic dye (e.g., D&C or FD&C) which has been precipitated onto an insoluble reactive or adsorptive substratum or diluent. The term “D&C” means drug and cosmetic colorants that are approved for use in drugs and cosmetics by the FDA. The term “FD&C” means food, drug, and cosmetic colorants which are approved for use in foods, drugs, and cosmetics by the FDA. Certified D&C and FD&C colorants are listed in 21 C.F.R. §74. 101 et seq. and include the FD&C colors Blue 1, Blue 2, Green 3, Orange B, Citrus Red 2, Red 3, Red 4, Red 40, Yellow 5, Yellow 6, Blue 1, Blue 2; Orange B, Citrus Red 2; and the D&C colors Blue 4, Blue 9, Green 5, Green 6, Green 8, Orange 4, Orange 5, Orange 1, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on.

Substrates suitable for forming lakes include, without limitation, mica, bismuth oxychloride, sericite, alumina, aluminum, copper, bronze, silver, calcium, zirconium, barium, and strontium, titanated mica, fumed silica, spherical silica, polymethylmethacrylate (PMMA), micronized teflon, boron nitride, acrylate copolymers, aluminum silicate, aluminum starch octenylsuccinate, bentonile, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonile, microcrystalline cellulose, rice starch, silica, talc, mica, titanium dioxide, zinc laurale, zinc myristate, zinc rosinate, alumina, attapulgite, calcium carbonate, calcium silicate, dexlran, nylon, silica silylate, silk powder, sericite, soy flour, tin oxide, titanium hydroxide, trimagnesium phosphate, walnut shell powder, and mixtures thereof.

Suitable lakes include, without limitation, those of red dyes from the monoazo, disazo, fluoran, xanthene, or indigoid families, such as Red 4, 6, 7, 17, 21, 22, 27, 28, 30, 31, 33, 34, 36, and Red 40; lakes of yellow pyrazole, monoazo, fluoran, xanlhene, quinoline, dyes or salt thereof, such as Yellow 5, 6, 7, 8, 10, and 11; lakes of violet dyes including those from the anthroquinone family, such as Violet 2 as well as lakes of orange dyes, including Orange 4, 5, 10, 11, and the like. Suitable Lakes of D&C and FD&C dyes are defined in 21 C.F.R. §82.51.

In one embodiment, a cosmetic composition as described herein comprises a total of about 0.1% to about 75% by weight of the particulate colorant component, based on the total weight of the composition. Typically, the particulate colorant component will comprise from about 0.5% to about 50% by weight, more typically from about 1% to about 40% by weight, and preferably from about 2% to about 30% by weight of the total composition. In other embodiments the particulate colorant component will comprise from about 3% to about 25% by weight, more typically from about 4% to about 15% by weight, and preferably from about 5% to about 10% by weight of the total composition.

### Cosmetically Acceptable Carrier

The compositions comprise a cosmetically acceptable carrier. By “cosmetically acceptable” is meant that the carrier is safe for contact with human skin. It is contemplated that any cosmetically acceptable carrier known in the art will be useful. The carrier may comprise water, hydrophobic, and/or hydrophilic solvents.

Suitable hydrophilic solvents include but are not limited to, water, isopropyl alcohol, ethyl alcohol, glycerin, butylene glycol, propylene glycol, pentylene glycol, caprylyl glycol, polyglycerol diisostearate, dimethylsiloxane/glycol copolymer, isopropyl myristate, triisostearyl citrate, or any combinations thereof. Suitable hydrophobic carriers include volatile or non-volatile hydrocarbon oils, silicones, fatty ester oils, and the like.

The compositions may comprise at least one high evaporation rate solvent in combination with at least one medium evaporation rate solvent and/or at least one slow evaporation rate solvent. As used herein, a high evaporation rate solvent may be characterized as a solvent that exhibits about 20% to about 40% weight loss al 35° C. over 60 minutes and/or about 40% to about 50% weight loss at 35° C. over 120 minutes. A medium evaporation rate solvent may be characterized as a solvent that exhibits about 10% to about 15% weight loss at 35° C. over 60 minutes and/or about 20% to about 30% weight loss at 35° C. over 120 minutes. A slow evaporation rate solvent may be characterized as a solvent that exhibits less than about 10% weight loss at 35° C. over 60 minutes and/or about 5% to about 15% weight loss at 35° C. over 120 minutes. Non-limiting example of high evaporation rate solvents include hexamethyl disiloxane and/or a silicone fluid having a viscosity of less than 1 cSt at 25° C., including, for example, those silicone fluids having a viscosity of 0.65 cSt. A non-limiting example of a medium evaporation rate solvent includes mixed dimethicones, e.g., a dimethicone/trisiloxane blend. Non-limiting examples of slow evaporation rate solvents include cyclopentasiloxane, methyl trimethicone, and isododecane.

The compositions of the invention may, in some embodiments, be provided as anhydrous formulations. By “anhydrous” is meant that the weight percentage of water in the composition is less than about 1% by weight. Preferably, the anhydrous compositions are substantially free of water by which is meant that water is not deliberately added to the compositions and the level of water is no more than would be expected based on the absorption of water from the air.

The carrier may comprise from about 5% to about 90% by weight of the composition, typically from about 30% and about 80% by weight, and more typically from about 50% and about 70% by weight of the composition.

### Other Ingredients

Shine Agents

The cosmetic compositions of the invention may optionally include one or more agents that provide or enhance shine. Shine enhancing agents will typically have a refractive index greater than about 1.4, preferably greater than about 1.5 when measured as a film at 25° C. Suitable shine enhancing agents include without limitation, polyols, fatty esters, silicone oils, phenylpropyldimethylsiloxysilicale, polybutene, polyisobutene, hydrogenated polyisobutene, hydrogenated polycyclopentadiene, propyl phenyl silsesquioxane resins; lauryl methicone copolyol, perfluorononyl dimethicone, dimethicone/trisiloxane, methyl trimethicone, and combinations thereof. In one embodiment, the composition will comprise a shine-enhancing agent in an amount from about 0.1% to about 10% by weight, more preferably from about 1% to about 5% by weight, based on the total weight of the composition.

Waxes

The cosmetic compositions may optionally include one or more waxes. The one or more waxes can be natural (e.g., vegetable, animal, or mineral) waxes or synthetic waxes (e.g., polyolefine, Fisher Tropsch, etc.). A preferred wax is microcrystalline waxes, which will preferably be composed of C₄ to C₅₀ hydrocarbons and will have a melting point preferably greater than about 60° C. Other waxes that may be mentioned include, without limitation, glyceryl iribehenale, candelilla, carnuaba, ozokerite, paraffin, polyethylene, beeswax, ceresin, hydrogenated castor oil, Japan wax, and mixtures thereof. In one embodiment, the amount of wax is less than about 2% of the total weight of the composition. In another embodiment, the amount of wax ranges from about 0.1% to less than about 2% by weight based on the total weight of the composition. However, more wax can be used if clarity is not a concern. For example, a lip stick may comprise wax from about 5% to about 25% by weight based on the weight of the composition.

Pigments and Fillers

The cosmetic compositions may optionally further comprise various other pigments, pearlescents, dyes, lakes, and fillers, as is customary in a given product. These include, without limitation, metal oxide pigment such as iron oxides and titanium dioxide, silica, alumina, nylon powder, Teflon powder, PMMA, silicone elastomers, and the like. For other pigments, lakes and dyes used in cosmetic industry, refer to the Cosmetic Ingredient Dictionary (INCI) and Handbook, 12th Edition (2008), published by the Cosmetic, Toiletry, and Fragrance Association (CTFA). Such additional pigments, fillers and the like will typically comprise from about 0.1% to about 20% by weight of the composition, more typically from about 0.8% to about 10% by weight of the composition.

Other Film Formers

In addition to the film formers of the invention, which act synergistically with the solvent system herein to provide a thickened transfer-resistant composition, other water-soluble, water-dispersible, or water-insoluble film formers, including film forming polymers, may be employed. The term film-forming polymer may be understood to indicate a polymer which is capable, by itself or in the presence of at least one auxiliary film-forming agent, of forming a continuous film which adheres to a surface.

Polymeric film formers include, without limitation, acrylic polymers or copolymers, acrylates, polyolefins, polyvinyls, polacrylates, polyurethanes, silicones, polyamides, polyethers, polyesters, fluoropolymers, polyethers, poly-acetals, polycarbonates, polyamides, polyimides, rubbers, epoxies, formaldehyde resins, organosiloxanes, dimethicones, methicones, cellulosics, polysaccharides, polyquaterniums, and the like. Suitable film formers include those listed in the Cosmetic Ingredient Dictionary (INCI Handbook, 12th Edition (2008)).

Emulsions

The compositions may be formulated as water-in-oil (W/0) emulsions, oil-in-water (0/W) emulsions, water-in-silicone, silicone-in-water emulsions, and the like. These emulsions comprise a continuous phase and a discontinuous phase. The continuous phase may be aqueous, oil-based, or silicone-based and the discontinuous phase may likewise be aqueous, oil-based, or silicone-based, depending on the nature of the continuous phase. Combined oil and silicone phases are also possible.

The oil phase may comprise any of the hydrophobic oils, including, without limitation, vegetable oils; fatty acid esters; fatty alcoholsl; isoparaffins such as isododecane; silicone oils such as dimethicones, cyclic silicones, and polysiloxanes; hydrocarbon oils such as mineral oil, petrolatum, isoeicosane and polyisobutene; natural or synthetic waxes; and the like.

The emulsions will typically comprise an amount of emulsifier sufficient to stabilize the emulsion. The amount of emulsifier will typically be from about 0.001% to about 20%, but preferably will range from about 0.01% to about 10%, and most preferably about 0.1% to about 5%, based upon the total weight of the composition.

Emollients

The cosmetic compositions may optionally comprise one or more emollients in an amount from about 0.1% up to about 20% by weight, based on the total weight of the composition. More typically, emollients will be present in an amount from about 2% to about 15%, preferably, about 5%. Emollients useful in the present invention include any known to the art, including, but not limited to, oils and esters, such as lanolin and petrolatum. Other emollients include jojoba oil, lanolin oil, coconut oil, palm kernel glycerides, grape seed oil, evening primrose oil, sesame oil, castor oil, meadowfoam seed oil, emu oil, dimethicone copolyol meadowfoamate, wheat germ oil, macadamia nut oil, avocado oil, and mixtures thereof.

Thickeners

The composition may comprise an additional thickener depending on the desired use of the composition. Suitable thickeners include those such as vegetable gums, carboxymethyl cellulose, silica, additional acrylic acid polymers, clays, such as hectorites, bentonites, hydrated magnesium and aluminum silicates, or calcium silicates, or the like. When present, thickeners will comprise from about 0.1% to about 15% by weight of the composition, more typically from about 1% to about 5% by weight of the composition.

### Other Ingredients

The compositions may include other ingredients such as one or more anesthetics, anti-allergenics, antifungals, anti-inflammatories, antimicrobials, antiseptics, chelating agents, emollients, emulsifiers, fragrances, humectants, lubricants, masking agents, medicaments, moisturizers, pH adjusters, preservatives, protectants, soothing agents, stabilizers, sunscreens, surfactants, thickeners, viscosifiers, vitamins, or any combinations thereof.

In addition, the compositions may comprise other ingredients and additives known in the art, depending on the purpose for which the cosmetic is intended. For example, a composition described herein may optionally include one or more functional agents, fillers and fragrances.

The compositions according to the invention may be useful in a variety of cosmetic and personal care products, including without limitation, lipsticks, and lip colors, lip gloss, mascaras, transfer-resistant foundations, eyeliner, eye shadow, water-proof sunscreens and insect repellents, skin care products, hair care products, antiperspirants and deodorants, and other cosmetic products where transfer resistant films are desired.

In one embodiment, the invention is formulated in a conventional lipstick or lip color product and may include, without limitation, any of the components disclosed in U.S. Pat. No. 6,509,009, U.S. Pat. No. 6,428,797, U.S. Pat. No. 6,261,576, U.S. Pat. No. 5,747,017, U.S. Pat. No. 5,318,775, and U.S. Pat. No. 4,935,228.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention, as are ranges based thereon. Where a combination is disclosed, each subcombination of the elements of that combination is also specifically disclosed and is within the scope of the invention. Conversely, where different elements or groups of elements are disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

The invention is illustrated by the following specific examples.

The examples below are formulated by mixing the silicone modified acryl resin (powder) with the coconut alkanes/coco-caprylate/caprate components to form a pre-mix. Then, the remaining ingredients are mixed together to form a cosmetic composition, such as a lipstick.

| | Percent by weight of | | | |
|---|---|---|---|---|
| Ingredients | composition | | | |
| Example | 1 | 2 | 3 | 4 |
| Silicone Modified Acryl Resin¹ | 40 | 42 | 45 | 40 |
| Coconut Alkanes/Coco-Caprylate/Caprate² | 25 | 26 | 25 | 20 |
| Trisiloxane | 16 | 20 | 17 | 20 |
| Isododecane | 5 | 0 | 0 | 0 |
| Wax | 4 | 3 | 5 | 4 |
| Dye | 2 | 2 | 2 | 2 |
| Disteardimonium Hectorite | 0.5 | 0.6 | 0.6 | 0.5 |
| Coco-Caprylate/Caprate | 0.4 | 0.4 | 0.4 | 0.5 |
| Phenoxyethanol | 0.4 | 0.4 | 0.4 | 0.4 |
| Polysilicone-11/Isododecane | 0 | 0 | 0 | 4 |
| Filler | q.s. | q.s. | q.s. | q.s. |

¹Granacrysil BAS available from Grant Industries
²Vegelight 1214LC available from Grant Industries

What is claimed is:

1. A transfer-resistant cosmetic composition comprising:
a) at least one film forming polymer;
b) at least one naturally derived volatile solvent; and
c) a carrier.

2. A cosmetic composition according to claim 1, wherein said film forming polymer is a silicone acrylate polymer.

3. A cosmetic composition according to claim 1, wherein said film forming polymer is selected from the group consisting of butyl acrylate/hydroxypropyl dimethicone acrylate copolymer, acrylates/dimethicone copolymer, acrylates/ethylhexyl acrylate/dimethicone methacrylate copolymer, and combinations thereof.

4. A cosmetic composition according to claim 1, wherein said alkanes solvent comprises coconut alkane and caprylate/caprate.

5. A cosmetic composition according to claim 1, further comprising a particulate colorant.

6. A cosmetic composition according to claim 5, wherein said particulate colorant is selected from the group consisting of a color extender, dye, pigment, lake, toner, and combinations thereof.

7. A cosmetic composition according to claim 1, wherein said volatile alkane is sourced from a vegetable oil feedstock.

8. A cosmetic composition according to claim 1, further comprising one or more ingredients selected from the group consisting of shine agents, waxes, pigments, fillers, additional film formers, emollients, thickeners, anesthetics, anti-allergenics, antifungals, anti-inflammatories, antimicrobials, antiseptics, chelating agents, emollients, emulsifiers, fragrances, humectants, lubricants, masking agents, medicaments, moisturizers, pH adjusters, preservatives, protectants, soothing agents, stabilizers, sunscreens, surfactants, thickeners, viscosifiers, vitamins, and combinations thereof.

9. A cosmetic composition according to claim 1, wherein said composition is in the form of an emulsion.

10. A cosmetic composition according to claim 1, wherein said polymer is present at a level of from about 0.1% to about 85% by weight of the composition.

11. A cosmetic composition according to claim 1, wherein said composition comprises less than about 10% by weight of the composition of one or more solvents selected from the group consisting of methyl trimethicone, isododecane, dimethylsiloxane, cyclodimethicone pentamer/hexamer, and combinations thereof.

12. A cosmetic composition according to claim 1, wherein said film-film forming polymer derived from acrylic acid and said at least one volatile alkanes solvent are formed in a pre-mix to formed a thickened system.
